(19) United States
(12) Patent Application Publication
(10) Pub. No.: US 2015/0140548 A1
(43) Pub. Date: May 21, 2015

(54) EXTRACTION CONTROL FOR RNA

(71) Applicant: Vela Operations PTE. Ltd., Singapore (SG)

(72) Inventor: Xin Zhang, Singapore (SG)

(73) Assignee: VELA OPERATIONS PTE. LTD., Singapore (SG)

(21) Appl. No.: 14/402,606

(22) PCT Filed: May 17, 2013
(86) PCT No.: PCT/IB2013/054032

§ 371 Date: Nov. 20, 2014

(30) Foreign Application Priority Data
May 21, 2012 (GB) 1208942.1

(51) Int. Cl. C12Q 1/68; C12Q 1/70

(52) U.S. Cl. CPC C12Q 1/6851; C12Q 1/701; C12Q 1/6806; C12Q 2600/166
USPC 435/5; 435/235.1

(57) ABSTRACT

The present invention relates to compounds for use in the control of extraction procedures, in particular in connection with nucleic acid material for use in PCR, in particular RT-PCR and more preferably real-time RT-PCR (quantitative PGR). The extraction control according to the present invention is based on plant virus material which can be produced at low cost, in large quantities and which has good stability.

This invention relates to the field of diagnostics, particularly to compounds serving as extraction controls in methods for the detection of the presence or absence of target nucleic acids, e.g. RNA, in a sample to be analyzed. Defined quantities of extraction controls of the invention are added to said samples and nucleic acids derived from such samples are subsequently analyzed in RT-PCR-based assays for the detection of target RNA. The present invention relates also to compositions, kits, assays, and articles of manufacture. comprising the extraction control of the invention as well as to methods for the extraction of nucleic acids and subsequent RT-PCR analysis, wherein the extraction controls are used.

### BACKGROUND ART

PCR is considered the most sensitive and rapid method for detecting nucleic acids of interest, e.g. nucleic acids derived from a pathogen in a particular sample. PCR is well known in the art and has been described in U.S. Pat. No. 4,683,195 to Mullis et al., U.S. Pat. No. 4,683,202 to Mullis, U.S. Pat. No. 5,298,392 to Atlas et al., and U.S. Pat. No. 5,437,990 to Burg et al.

For the PCR step, oligonucleotide primer pairs specific for each of the target nucleic acid are provided wherein each primer pair includes a first nucleotide sequence complementary to a sequence flanking the 5′ end of the target nucleic acid sequence and a second nucleotide sequence complementary to a nucleotide sequence flanking the 3′ end of the target nucleic acid sequence. The nucleotide sequences of each oligonucleotide primer pair are specific to particular nucleic acid target and should not cross-react with other nucleic acids.

Reverse transcription (RT)-PCR is currently the method of choice for the detection of target ribonucleic acids, e.g. nucleic acids whose presence or absence may be detected in diagnostic applications, for example in the diagnosis of a viral infection, the presence of biological contaminants in water, food, etc.

RT-PCR and in particular real-time RT-PCR or qRT-PCR (quantitative RT-PCR, which has been described in various textbooks, e.g., in Logan J, Edwards K, Saunders N (editors), (2009). Real-Time PCR: Current Technology and Applications. Caister Academic Press), can be used in the detection of RNA of pathogens that do not contain DNA, such as HIV, hepatitis A virus (HAV), influenza virus, etc., but it may also be used in the detection of RNA synthesized on the basis of DNA templates (cellular mRNA, viral transcripts, microRNA, bacterial or fungal transcripts, etc.).

When RT-PCR is used in diagnostic applications, the accuracy of a diagnosis not only depends on the integrity of chemical compounds used for the amplification of target nucleic acid sequences, but also on the availability of nucleic acid sequences successfully extracted from a sample.

The analysis of biological specimens, e.g. blood samples, stool samples, contaminated water, food, etc., can be influenced by diverse chemical and physical factors that can affect the integrity of nucleic acids present in such samples.

Examples for such factors are those playing a role in storage and retrieval conditions of samples (temperature, pH, time that has elapsed between obtaining the sample and extracting the nucleic acids therein, etc.). Moreover, nucleic acids present may be affected by the presence of potentially interfering factors in a sample, e.g. inhibitory proteins or nucleic acid degrading enzymes (RNAses, DNAses, etc.). Failure to extract intact or sufficient amounts of nucleic acids from a sample may also be due to bad quality of reagents used in the extraction procedure.

One or more factors exerting a negative influence on the result of the extraction of target nucleic acids that were originally present in a sample may be responsible for the failure of their detection. Therefore, when RT-PCR is performed using nucleic acid material extracted from a sample, failure to amplify a target nucleic acid may be incorrectly interpreted as the absence of target nucleic acids. In the case of diagnostic applications, for instance in the detection of pathogen-derived RNA or of oncogene transcripts, it is therefore of utmost importance to include controls for the reliability of the extraction procedure. In the absence of appropriate extraction controls, failure to detect a target nucleic acid may be interpreted as false negative test result. This may have serious consequences as the results may the cause for an incorrect diagnosis and subsequently the wrong treatment of the source organism, e.g. a human patient.

It is also important that extraction controls have a good long-term storage stability (i.e. that the extraction control can be used for at least one year, preferably two years or longer after shipping) at relatively unfavorable conditions, e.g. elevated temperatures such as temperatures above 4° C., preferably above 20° C., and that the extraction controls can be subjected to harsh extraction conditions without fear of their degradation. Furthermore, extraction controls should be safe under laboratory conditions and represent no threat to environment or health of personnel working herewith.

The present invention relates to such improved extraction controls in methods aiming at detecting the presence or absence of target RNA in various sources. In particular, the present invention relates to the use of tobacco mosaic virus (TMV) particles (virions) as source of control RNA, and as an extraction control reagent for PCR-based detection methods.

TMV, which belongs to the genus tobamovirus, is one of the best characterized viruses in the world. It is responsible for infection of plants, e.g. tobacco. Other tobamoviruses infect, e.g., tomatoes, pepper and eggplant. Due to its structure, TMV virions are extremely stable and remain infectious for many years in vitro (http://www.ncbi.nlm.nih.gov/ICTVdb). TMV belongs to positive single-strand RNA viruses and has a genome of about 6,400 bases. TMV does neither infect animals, in particular mammals such as humans, microorganisms nor microorganims.

The present inventors have surprisingly found that TMV virions can serve as ideal extraction controls for protocols aiming directed to the extracting RNA from a sample, which can be used in real-time RT-PCR (qRT-PCR) reactions.

The use of TMV has many advantages. TMV is easy to produce in large quantities without the need for complicated cell culture techniques that are prone to contaminations and cause substantial costs. Cell culture, cloning of extraction controls, transcription, purification of the control material, etc. are very time-consuming, e.g., when using Encephalomyocarditisviruses such as Mengoviruses as extraction controls (WO2008/145197). Furthermore, as Mengoviruses are RNA viruses having a larger genome than, e.g., TMV, and as they are able to infect animal cells, safety measures are considerable even when working with non-pathogenic mutant strains as back-mutations cannot be ruled out and may restore the pathogenic potential.

TMV has also the advantage of being highly stable in extraction control media. The viruses can be produced in large amounts at low cost. Routine measures in laboratories for the treatment of waste material avoid any risks of contamination of the environment with such extraction control material. The infectivity of TMV can be controlled through simple hygienic measures, for example, a pasteurization step at temperatures of about 90° C. for 1 hour leads to a complete inactivation of TMV virus (Forschungsberichtsblatt zum Forschungsvorhaben “Untersuchungen zur Seuchen-und Phytohygiene in Anaerobanlagen PUGU 98009”) so that the risk of infectious viral material leaving a laboratory does not represent a problem.

Alternatively, instead of TMV, another positive single-stranded plant virus such as Tobacco Rattle Virus (TRV) may be used. One advantage of using TRV is that it requires nematodes such as Trichodorus or Paratrichodorus to transfer the virus from one plant to another and is therefore easily controllable.

### DESCRIPTION OF THE INVENTION

In a first embodiment, the present invention relates to processes or methods for the detection and/or quantification of at least one target nucleic acid in a biological sample, wherein the method comprises a nucleic acid extraction step in the presence of a plant virus added to said sample before extraction is carried out. Herein below, the addition of extraction control of the present invention to a biological sample before extraction of RNA is also referred to as “spiking”.

In a preferred embodiment of the invention, the quantification and/or detection process of target RNA comprises RT-PCR, preferably real time RT-PCR (also referred to as “quantitative RT-PCR”, qRT-PCR).

A biological sample may include a sample obtained from a water supply; sewer treatment area; a soil sample from a farming area; animal grazing area; waste disposal area; and/or a sample obtained from virtually any water source used by animals or humans for consumption, cleaning, or any other domestic or commercial use; or the like. In addition, a biological sample may comprise human or animal waste materials (e.g., stool), medical refuse (bandages and wound dressings), body fluid (urine, plasma, blood, mucus, etc), and/or the like. In some embodiments, the methods provide for the screening and/or testing of a biological specimen such as drinking water and/or bodies of water (such as a stream, river, or lake) from which drinking water is obtained.

In further embodiments of the invention, the extraction of nucleic acids (e.g. RNA) can be performed using any method known in the art (cf. “Molecular Cloning: A Laboratory Manual”, Third and Fourth Editions; Sambrook et al.) or using kits available on the market from various commercial sources such as Qiagen (Hilden, DE) or Macherey-Nagel (DE).

In further embodiments of the invention, the plant virus is a positive single-strand RNA virus, preferably selected from the genuses tobamovirus, topravirus, etc. In particularly preferred embodiments of the invention, the virus is TMV.

In preferred embodiments of the invention, the target RNA is of viral, bacterial, fungal or parasitic origin. In other embodiments of the invention, the target RNA is the transcription product of cellular genes of an organism from which the sample was obtained, e.g. RNA splice forms involved in diseases, RNA transcribed in response to infections, etc.

In yet other embodiments of the invention, methods or processes of the invention are directed to the amplification of target nucleic acids comprising the steps:
a) obtaining a sample suspected to contain a target nucleic acid (e.g. RNA);
b) extracting nucleic acids (e.g. RNA) from said sample in the presence of a defined amount of an extraction control of the present invention, preferably in the range of 1 fg to 1000 fg, preferably 10 fg to 100 fg, wherein said extraction control is added to the sample suspected to contain at least one nucleic acid molecule of interest/to be analyzed prior to extraction of nucleic acids;
c) reverse transcription of RNA extracted from said sample to produce cDNA;
d) amplification of target nucleic acids using at least one reaction mixture comprising primers specifically hybridizing with a target nucleic acid molecule and/or a primer pair specifically hybridizing with nucleic acids derived from the extraction control and/or probes, wherein said primers and/or probes carry fluorescent moieties allowing their detection;
e) monitoring the amplification process and/or detecting and quantifying the amount of products;
f) optionally calculating the amount of amplification products derived from both, extraction control nucleic and target nucleic acids;
g) optionally deciding, based on the results obtained in the preceding steps, on the therapeutic treatment of a patient (e.g. administration of anti-viral, anti-bacterial, anti-fungal drugs, etc.).

The present invention relates also to compositions for use in the extraction of nucleic acids comprising an extraction control, said extraction control comprising a plant virus. Said plant virus is preferably selected from RNA viruses, such as viruses of the family of virgaviridae, is preferably a positive single-stranded virus, for example a virus selected from the genera tobamovirus, tobravirus, or the like, e.g. TMV or TRV (tobacco rattle virus).

In yet additional embodiments, the invention is directed to kits comprising an extraction control as defined in the above sections. Preferably, the kits according to the present invention are diagnostic kits, wherein the diagnostic kits comprise components for performing real-time PCR. The kits according to the present invention comprise an extraction control having a storage stability of at least one year, preferably 2, 3, 4, 5, 10 years at ambient temperature. In some embodiments of the invention, the kit components may be freeze-dried and may contain a buffer for reconstitution.

In the processes and/or methods of the invention, in the compositions or kits or other products according to the invention, the plant virus has been inactivated, i.e. the pathogenicity is reduced. In preferred embodiments, the inactivation is achieved by UV-treatment of the virus particles, e.g. after purification from infected plant material. UV-treatment is a physicochemical inactivation method that leaves viral particles intact as it affects the structure of viral proteins only minimally, but it crosslinks nucleic acids. Traditionally, UV-treatment is not used in methods for the analysis of nucleic acids, but for protein-based assays such as ELISA, Western Blot and other protein-based assays. It was surprisingly found that UV-treatment can reduce the pathogenicity of TMV, whereas the amount of RNA extracted from UV-treated virions is still sufficiently high and the integrity of the RNA of such good quality to use the same in qRT-PCR reactions. It was surprising to note that UV-irradiated plant viruses such as TMV can still be used as controls for RNA-based assays.

A defined amount of UV-inactivated plant-virus extraction control that is added to the reactions described herein ranges from about 1 fg to about 1000 fg or more, e.g. 1500 fg, 2000 fg or 2500 fg, preferably from 5 fg to 500 fg, more preferably from 5 fg to 250 fg, still more preferably from 10 fg to 100 fg and most preferably from 10 fg to about 75 fg.

The present invention pertains also to an assay for the diagnosis of the presence or absence of a target RNA molecule. Said assay comprises an extraction control as defined in any of the previous sections.

Furthermore, the present invention relates to automated devices for the extraction of nucleic acids from a biological sample comprising a source, e.g. a compartment of the device containing the extraction control of the invention.

Commercial uses of the present methods include clinical diagnosis of a human specimen, veterinary diagnosis from an animal specimen, water quality testing from recreational or drinking water samples, food sample testing, and environmental testing from soil or other sample types as defined above.

Compositions, components of kits or other products of the invention may be prepared in lyophilized form and/or provided in one or more master mixes optionally comprising additional components, e.g., for the extraction of nucleic acids, for performing reverse transcription and/or PCR.

In some aspects of the invention, the primers and/or probes of the invention can be labeled with a fluorescent moiety. Fluorescent moieties for use in real-time PCR detection are known to persons skilled in the art and are available from various commercial sources, e.g. from life Technologies™ or other suppliers of ingredients for real-time PCR.

In addition, the methods and products of the present invention may include a positive internal control for the PCR as known in the art or as described in GB application GB1204776.7. The term “internal control” as used herein refers to a nucleic acid sequence that may be used to demonstrate that a PCR reaction is functioning to detect a nucleic acid sequence.

In some aspects of the invention, there are provided articles of manufacture, or kits. Articles of manufacture can include fluorophoric moieties for labeling the primers or probes or the primers and probes are already labeled with donor and corresponding acceptor fluorescent moieties.

Amplification generally involve the use of a polymerase enzyme. Suitable enzymes are known in the art, e.g. Taq Polymerase, etc.

As used herein, the term “probe” or “detection probe” refers to an oligonucleotide that forms a hybrid structure with a target sequence contained in a molecule (i.e., a “target molecule”) in a sample undergoing analysis, due to complementarity of at least one sequence in the probe with the target sequence. The nucleotides of any particular probe may be deoxyribonucleotides, ribonucleotides, and/or synthetic nucleotide analogs.

The term “primer” or “amplification primer” refers to an oligonucleotide that is capable of acting as a point of initiation for the 5′ to 3′ synthesis of a primer extension product that is complementary to a nucleic acid strand. The primer extension product is synthesized in the presence of appropriate nucleotides and an agent for polymerization such as a DNA polymerase in an appropriate buffer and at a suitable temperature.

The most widely used target amplification procedure is PCR, first described for the amplification of DNA by Mullis et al. in U.S. Pat. No. 4,683,195 and Mullis in U.S. Pat. No. 4,683,202 and is well known to those of ordinary skill in the art. Where the starting material for the PCR reaction is RNA, complementary DNA (“cDNA”) is made from RNA via reverse transcription. A PCR used to amplify RNA products is referred to as reverse transcriptase PCR or “RT-PCR.”

Where the starting material for the PCR reaction is RNA, complementary DNA (“cDNA”) is synthesized from RNA via reverse transcription. The resultant cDNA is then amplified using the PCR protocol described above. Reverse transcriptases are known to those of ordinary skill in the art as enzymes found in retroviruses that can synthesize complementary single strands of DNA from an mRNA sequence as a template. A PCR used to amplify RNA products is referred to as reverse transcriptase PCR or “RT-PCR.”

The terms “real-time PCR” and “real-time RT-PCR,” refer to the detection of PCR products via a fluorescent signal generated by the coupling of a fluorogenic dye molecule and a quencher moiety to the same or different oligonucleotide substrates. Examples of commonly used probes are TAQMAN® probes, Molecular Beacon probes, SCORPION® probes, and SYBR® Green probes. Briefly, TAQMAN® probes, Molecular Beacons, and SCORPION® probes each have a fluorescent reporter dye (also called a “fluor”) attached to the 5′ end of the probes and a quencher moiety coupled to the 3′ end of the probes. In the unhybridized state, the proximity of the fluor and the quencher molecules prevents the detection of fluorescent signal from the probe; during PCR, when the polymerase replicates a template on which a probe is bound, the 5′-nuclease activity of the polymerase cleaves the probe thus, increasing fluorescence with each replication cycle. SYBR Green® probes binds double-stranded DNA and upon excitation emit light; thus as PCR product accumulates, fluorescence increases. In the context of the present invention, the use of TAQMAN® probes is preferred. When real-time RT-PCR is used, it is possible to measure or determine also the quantity of the target nucleic acid in the sample. In the latter case, the real-time RT-PCR is often referred to as qRT-PCR.

The terms “fluorophore”, “fluorogenic dye”, “fluorescent dye” as used herein designate a functional group attached to a nucleic acid that will absorb energy of a specific wavelength and re-emit energy at a different, but equally specific, wavelength.

The terms “complementary” and “substantially complementary” refer to base pairing between nucleotides or nucleic acids, such as, for instance, between the two strands of a double-stranded DNA molecule or between an oligonucleotide primer and a primer binding site on a single-stranded nucleic acid to be sequenced or amplified. Complementary nucleotides are, generally, A and T (or A and U), and G and C. Within the context of the present invention, it is to be understood that the specific sequence lengths listed are illustrative and not limiting and that sequences covering the same map positions, but having slightly fewer or greater numbers of bases are deemed to be equivalents of the sequences and fall within the scope of the invention, provided they will hybridize to the same positions on the target as the listed sequences. Because it is understood that nucleic acids do not require complete complementarity in order to hybridize, the probe and primer sequences disclosed herein may be modified to some extent without loss of utility as specific primers and probes. Generally, sequences having homology of about 90% or more fall within the scope of the present invention. As is known in the art, hybridization of complementary and partially complementary nucleic acid sequences may be obtained by adjustment of the hybridization conditions to increase or decrease stringency, i.e., by adjustment of hybridization temperature or salt content of the buffer.

The term “hybridizing conditions” is intended to mean those conditions of time, temperature, and pH, and the necessary amounts and concentrations of reactants and reagents, sufficient to allow at least a portion of complementary sequences to anneal with each other. As is well known in the art, the time, temperature, and pH conditions required to accomplish hybridization depend on the size of the oligonucleotide probe or primer to be hybridized, the degree of complementarity between the oligonucleotide probe or primer and the target, and the presence of other materials in the hybridization reaction admixture. The actual conditions necessary for each hybridization step are well known in the art or can be determined without undue experimentation.

The term “label” as used herein refers to any atom or molecule that can be used to provide a detectable (preferably quantifiable) signal, and that can be attached to a nucleic acid or protein via a covalent bond or noncovalent interaction (e.g., through ionic or hydrogen bonding, or via immobilization, adsorption, or the like). Labels generally provide signals detectable by fluorescence, chemiluminescence, radioactivity, colorimetry, mass spectrometry, X-ray diffraction or absorption, magnetism, enzymatic activity, or the like. Examples of labels include fluorophores, chromophores, radioactive atoms, electron-dense reagents, enzymes, and ligands having specific binding partners.

In another embodiment of the invention, the extraction control of the present invention is found in a compartment of a device that is suitable in fully automated laboratories capable of extracting nucleic acids from a sample, optionally capable of reverse transcription methods using the previously extracted nucleic acids, setting up amplification reactions, and performing said amplification reactions (e.g. qRT-PCR) using the components described herein and quantitatively and/or qualitatively detecting nucleic acid targets, e.g. using real-time PCR.

In a further aspect, the present invention relates to a composition comprising primers and probes. Preferably, the composition comprises also ingredients, e.g. enzymes, buffers and deoxynucleotides necessary for reverse transcription and/or PCR, preferably for qualitative and/or quantitative RT-PCR. The composition may be stored in the refrigerator in a liquid state or deep-frozen in a suitable medium, or it may be lyophilized and reconstituted before use and which may further comprises detectable probes and/or an internal control.

The term “amplification” of nucleic acids, including DNA, as used herein means the use of PCR to increase the concentration of a particular nucleic acid sequence within a mixture of nucleic acid sequences. The term “PCR” as used herein means the polymerase chain reaction, as is well-known in the art. The term includes all forms of PCR, such as, e.g., real-time PCR and quantitative PCR.

The particular nucleic acid sequence that is amplified is described herein as a “target” sequence. The term “target” sequence as used herein means the sequence of a nucleic acid that is amplified by PCR. The term “target” nucleic acid sequence as used herein means the sequence of a nucleic acid that is amplified by PCR. Preferably, the target is RNA and the nucleic acid amplification protocol comprises RT-PCR.

The terms “biological sample” and “sample” as used herein mean any specimen or sample of matter capable of containing an organism. Non-limiting examples include a sample of water, a soil sample, an air sample, a stool sample, a urine sample, and the like. In other embodiments of the invention, the sample is a tissue fluid from a patient, which may be selected from the group consisting of blood, plasma, serum, lymphatic fluid, synovial fluid, cerebrospinal fluid, amniotic fluid, amniotic cord blood, tears, saliva, and nasopharyngeal washes.

The term “patient” as used herein is meant to include both human and veterinary patients.

The terms “pathogen,” “organism,” and “species” are used interchangeably herein and refer to any one species, or closely-related group of species, that may be uniquely identified by an oligonucleotide sequence. The species may be known or unknown and may include any type of virus, bacterium, fungus, etc.

The term “primer pair” as used herein means a pair of oligonucleotide primers that are complementary to the sequences flanking a target sequence. The primer pair consists of a forward primer and a reverse primer. The forward primer has a nucleic acid sequence that is complementary to a sequence upstream, i.e. 5′ of the target sequence. The reverse primer has a nucleic acid sequence that is complementary to a sequence downstream, i.e. 3′ of the target sequence.

The terms “probe” and “probe pair” refer to one or two oligonucleotide sequences that are complementary to a specific target sequence and are covalently linked to a fluorophore. A probe pair includes two oligonucleotides: a “donor probe” and an “acceptor probe.” When both probes are bound to the target sequence, the donor probe's fluorophore may transfer energy to the acceptor probe's fluorophore in a Förster resonance energy transfer (FRET).

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It also is be noted that as used herein and in the appended claims, the singular forms “a,” “an,” and “the” include the plural reference unless the context clearly dictates otherwise. This, for example, a reference to “a capsule” is a reference to one or more capsules and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein.

Any numerical values recited herein include all values from the lower value to the upper value in increments of one unit provided that there is a separation of at least two units between any lower value and any higher value. As an example, if it is stated that the concentration of a component or value of a process variable such as, for example, size, temperature, pressure, time and the like, is, for example, from 1 to 90, specifically from 20 to 80, more specifically from 30 to 70, it is intended that values such as 16 to 85, 22 to 68, 43 to 51, 30 to 32 etc., are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

Particular methods, devices, and materials are described, although any methods and materials similar or equivalent to those described herein can be used in the practice or testing of the invention. All references referred to herein are incorporated by reference herein in their entirety.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will be decisive.

The details of one or more embodiments of the invention are set forth in the description below. Other features, objects, and advantages of the invention will be apparent from the description, and from the claims.

### EXAMPLES

### Example 1

### TMV Inactivation Test

The following steps were performed:
200 μl TMV virus stock [purified manually from TMV infected Nicotiana benthamiana leaves](2.6 μg/μl) was added into a small Petri dish.
The virus stock was diluted with inoculation buffer (0.1 M phosphate buffer, pH 7.2) to make two stocks of TMV at 1.3 μg/μl.
A UV-crosslinker (Hoefer, UVC 500 UV crosslinker) was set at 0.72 J/cm2 and one TMV stock (obtained in step 2.) was subjected to UV-treatment.
UV-inactivated TMV was diluted to prepare solutions of 1 μg/40 μl, 10 μg/40 μl and 100 μg/40 μl.
Wildtype TMV stock was diluted to prepare solutions of 1 μg/40 μl, 100 ng/40 μl and 10 ng/40 μl.
Spray leaves of Nicotiana Benthamiana plant with sand powder to induce wound formation.
Inoculate two leaves of each Nicotlana Benthamiana plant with 40 μl of the respective diluted solutions. Each concentration of wild-type and UV-inactivated virus is tested on four pots of plants.
Four additional plants were inoculated with inoculation buffer as control.
The inoculated leaves were washed with water to remove the sand powder.
Plants were left in the dark overnight.
Plants were investigated for signs and symptoms of TMV infection.
Both, UV-inactivated TMV (1.3 μg/μl) and wild type TMV were diluted with inoculation buffer to obtain concentrations of 130 ng/μl and 13 ng/μl. 2 μl of each diluted wild-type and UV-inactivated TMV were used to extract the virus RNA by QIAamp Viral RNA Mini kit. Each of the extractions was performed in triplicate to identify the RNA recovery efficiency of irradiated TMV after extraction. The reason is that RNA may be destroyed after UV inactivation or RNA is cross-linked to each other or the proteins making it more difficult to extract RNA from the virus particles.
The same volumes of the RNA were used as templates in Taq Man real-time PCR by TMV primers and probe using Quanti-tech One step RT Mastermix in a Rotor-gene Q machine. The CT values were compared. The primer and probe concentrations were 200 nM.

1.1 Analysis of Nicotiana Benthamiana Plants Inoculated with UV-Inactivated and Non-Treated TMV

TABLE 1

| | Inoculated amount (Original stock | | |
|---|---|---|---|
| Virus used | concentration 2.6 mg/ml) | | Expectation |
| Inactivated | 100 μg | 1 μl | Show symptoms |
| TMV 0.72 | 10 μg | 10 μl | Show symptoms |
| J/cm² | 1 μg | 100 μl | No symptoms |
| Wild Type | 1 μg | 38.4 μl of stock + 161.6 μl | All show |
| | | of buffer | symptoms |
| | 0.1 μg | 20 μl of 100 μg stock + 180 | |
| | | μl of buffer | |
| | 0.01 μg | 20 μl of 10 μg stock + 180 | |
| | | μl of buffer | |

| | | |
|---|---|---|
| Negative | — | No symptoms |
| Control | | |
| (Inoculation | | |
| buffer) | | |

As can be derived from Table 1, an amount of 1 μg/100 μl UV-irradiated TMV virions in inoculation buffer did not induce symptoms after inoculation of sand powder-treated Nicotiana Benthamiana plants kept in the dark over night. At higher concentrations (100 μg/1 μl and 10 μg/10 μl) induced symptoms visible with the naked eye in such plants. Furthermore, TMV virions that were not UV-treated induced symptoms in plants at all concentrations tested (i.e. 1.0 μg/200 μl buffer, 0.1 μg/200 μl buffer or 0.01 μg/200 μg buffer). Inoculation buffer alone did not induce any symptoms. These results show that the treatment of TMV virions with UV at 0.72 J/cm² affected the RNA in these particles to such extent that substantially larger quantities of virus were necessary to induce symptoms in inoculated plants.

### 1.2 Analysis of the Effect of UV-Treatment on the Integrity of RNA

Real-Time PCR was used to determine the usefulness of RNA extracted from TMV particles previously added to samples and intact TMV particles, which were spiked in the negative control (NC) reactions and positive control (PC) reactions, respectively. The addition of intact TMV virions to the NC and PC reactions is not problematic as the RT-PCR amplification method involves cycle temperatures at which virions will denature and viral RNA will be released and be accessible to reagents for RT-PCR.

### Reagents:

Raw Tobacco Mosaic Virus (130 ng/μl)
UV inactivated Tobacco Mosaic Virus (0.72 J/cm²), (concentration: 130 ng/μl)
QIAamp Viral RNA Mini Kit (Lot No: 139294071)
Quantitech one step RT Master Mix (Lot No: 1032630)
RNase/DNase-free water (1^{st} Base, Biotechnology Grade, Cat. No: BUF-1180)
TMV Sense 2668: GACGTGCGAATTCCTCAG (SEQ ID NO: 1—I will prepare the sequence listing at the end). Synth ID-1076534. Order ID: 130658, 1^{st} BASE.
Primers (stock concentration: 10 mM), diluted with 1xTE buffer)
TMV Antisense 2812: GTTGATGTTGCATACTGGTTG (SEQ ID NO: 2). SynthID-1077701. OD: 130827, 1^{st} BASE. Primers (1^{st} Base,) (stock concentration: 10 mM, diluted with 1xTE buffer)
TMV Probe Antisense 2776: AACCTCTTGAACTGACAGCGTGTGC (SEQ ID NO: 3) (Probes labeled with CY5)

### Experimental Protocol

RNA was extracted from non-treated TMV (130 ng/μl, 13 ng/μl) and UV-inactivated TMV (130 ng/μl, 13 ng/μl), RNA was eluted with 60 μl elution buffer.

TABLE 2

| WT TMV | WT TMV | UV TMV | UV TMV |
|---|---|---|---|
| 130 ng/μl | 13 ng/μl | 130 ng/μl | 13 ng/μl |
| 4.33 ng/μl after | 0.433 ng/μl after | 4.33 ng/μl after | 0.433 ng/μl after |
| extraction | extraction | extraction | extraction |

A PCR master mix was produced and PCR reactions set up. The total volume is 25.00 pd per reaction. The PCR program consisted of the following steps: Step 1 was performed at 50° C. for 20 min; Step 2 was performed at 95° C. for 15 min; Step 3 was performed at 94° C. for 45 s and Step 4 was conducted at 60° C. for 75 s. Steps 3 and 4 were repeated 44 times. RT-PCR reactions were performed in Rotor Gene Q (Qiagen).

TABLE 3

| Component | Volume per reaction |
|---|---|
| HawkZ05 Master Mix (2.3x) | 10.87 μl |
| Mn (OAc)₂ (25 mM) | 1.50 μl (final: 1.5 mM) |
| Primers and probes mixture | 7.63 μl (final: 400 + 200 nM) |
| Template | 5.00 μl |

TABLE 4

| | | | | | To be spiked | |
|---|---|---|---|---|---|---|
| | | Aver- | RNA | Virus | in per | 24 × 4 |
| | | age | recov- | Inacti- | reaction | reaction |
| TMV | Conc. | CT | ery | vation | (minumum) | (minimum) |
| WT 1 | 13 ng | 18.17 | 100 | | 13 fg | 1.3 pg/kit |
| Inacti- | 13 ng | 19.85 | 30.8% | 99.6- | 13 fg | 1.3 pg/kit |
| vated 1 | | | | 99.9% | | |
| WT 1 | 130 ng | 15.29 | 100 | | 13 fg | 1.3 pg/kit |
| Inacti- | 130 ng | 16.90 | 30.8% | 99.6- | 13 fg | 1.3 pg/kit |
| vated 1 | | | | 99.9% | | |

The UV inactivated TMV included in one PCR reaction (13 fg) or in one kit (200 times 13 fg) is significantly lower than the lower limit of infection required for infection of Nicotiana Benthamiana, which is 2.6 μg.

### 1.3 Usefulness of TMV as EC in Clinical Assay

An RT-PCR assay was performed to determine, if spiking of TMV as EC in clinical samples will affect the detection of Influenza B when using an assay suitable for detection of Influenza A (strains H1 and H3) and Influenza B.

### Materials and Methods

### Rotor Gene Q (Qiagen, Machine 3 S/N: R1211131);

### HawkZ05 Fast 1-Step RT-PCR Lyo kit w/o ROX (Roche, HawkZ05 2.3×L/N:

### JGFE12A, Mn(OAc)₂ L/N: SZ0C11A);

Template: Positive control (PC) template (Flu B) and Flu B infected clinical samples;

### Primers and Probes Mix

TABLE 5

| | 1 Reaction | 54 Reactions |
|---|---|---|
| Components | (μl) | (μl) |
| HawkZ05 2.3x | 10.87 | 586.98 |
| Mn (OAc)₂ | 1.5 | 81 |
| Primers (Final Concentration: 400 | 7.63 | 412.02 |
| nM + 400 nM) and Probes (Final | | |
| Concentration: 200 nM) | | |
| Template | 5 | — |

Total RNA is extracted from Influenza B infected clinical nasal swab samples with/without 5 μl of 13 μg/μl TMV spiked in directly into the PCR tube. An RT-PCR master mix was prepared and 5 μl of the extracted RNA or the PC RNA was added to the PCR tube. The PCR protocol consisted of 5 min at 60° C., 5 min at 65° C., and 45 cycles at 94° C. for 5 seconds and 30 seconds at 60° C.

TABLE 6

| | | Channels | | | | | |
|---|---|---|---|---|---|---|---|
| | | Green | Yellow | Orange | Red | | |
| | | (H3) | (H1) | (Flu B) | (TMV) | Rep | Rep Ct |
| No | Name | Ct | Ct | Ct | Ct | Ct | Std Dev |

| | | | | | |
|---|---|---|---|---|---|
| 35 | Kit#2: PvP | 23.47 | | 23.47 | 0.01 |
| | 2771 (1/2) | | | | |
| 36 | Kit#2: PvP | 23.48 | | | |
| | 2771 (1/2) | | | | |
| 39 | Kit#2: PvP | 23.11 | 29.24 | 23.16 | 0.07 |
| | 2771 (1/2) + | | | | |
| | TMV 5 μl | | | | |
| 40 | Kit#2: PvP | 23.21 | 29.17 | | |
| | 2771 (1/2) + | | | | |
| | TMV 5 μl | | | | |
| 47 | Kit#2: | | | | |
| | UTM^{#} only | | | | |
| 48 | Kit#2: | | | | |
| | UTM only | | | | |
| 51 | Kit#2: Flu | 25.7 | | 25.68 | 0.03 |
| | B (−4) PC | | | | |
| 52 | Kit#2: Flu | 25.66 | | | |
| | B (−4) PC | | | | |

^{#}UTM = Universal Transport Medium

In the RT-PCR reaction Influenza A virus strains H1 and H3, respectively, were used as negative controls. No amplification products were detected for Influenza A. On the other hand, Influenza B was always detected with the respective kits used. TMV was also always detected when added to the sample. It was clearly seen that the presence of TMV did not negatively influence the detection of Influenza B in a 4-plex TaqMan real-time PCR.

1. A method for the detection and/or quantification of target nucleic acids in a sample, said method comprising:
a) extracting said target nucleic acids in the presence of a plant virus added to said sample prior to extracting;
b) amplifying said target nucleic acid; and
c) detecting and/or quantifying the target nucleic acids.

2. The method according to claim 1, wherein a defined amount of plant virus is added to the sample.

3. The method according to claim 1, further comprising performing RT-PCR.

4. The method according to claim 1, wherein the plant virus is a single-stranded positive RNA virus.

5. The method according to claim 1, wherein the single-stranded positive RNA virus is selected from the genus tobamovirus or the genus topravirus.

6. The method according to claim 1, wherein the plant virus is tobacco mosaic virus or tobacco rattle virus.

7. The method according to claim 1, wherein the RT-PCR method is real-time RT-PCR.

8. The method according to claim 1, wherein the target nucleic acid is RNA.

9. The method according to claim 8, wherein the RNA is pathogen-derived RNA or RNA derived from the donor of the sample.

10. The method according to claim 9, wherein the pathogen-derived RNA is of viral, bacterial, fungal or parasitic origin.

11. A method for the amplification of target nucleic acids comprising:
a) obtaining a sample suspected to contain a target nucleic acid;
b) extracting nucleic acids from said sample in the presence of a plant virus as extraction control, wherein said extraction control is added to the sample prior to extracting the nucleic acids;
c) optionally transcribing RNA by reverse transcription of RNA extracted from said sample;
d) amplifying said target nucleic acids using an amplification reaction mixture comprising primers specifically hybridizing with said target nucleic acid molecule and a primer pair specifically hybridizing with nucleic acids derived from the extraction control; and
e) quantifying the amplification products.

12. The method of claim 11, comprising performing real-time PCR.

13. The method of claim 11, wherein said plant virus is a single-stranded positive RNA virus.

14. The method according to claim 11, wherein said target nucleic acid is pathogen-derived RNA or RNA derived from the donor of the sample 9.

15. A composition comprising the extraction control, of claim 11.

16. The composition according to claim 15, wherein the plant virus is selected from RNA viruses.

17. The composition according to claim 15, wherein the plant virus is a positive single-stranded virus.

18. The composition according to claim 15, wherein said plant virus is tobacco mosaic virus.

19. A kit comprising the extraction control of claim 11.

20. The kit according to claim 19, wherein the kit is a diagnostic kit.

21. The kit according to claim 20, wherein the diagnostic kit is for PCR, RT-PCR, or real-time RT-PCR.

22. The kit according to claim 19, wherein the kit further comprises reagents for the extraction of nucleic acids.

23. The kit according to claim 19, wherein the kit comprises reagents for the extraction of RNA.

24. The kit according to claim 19, wherein the extraction control has a storage stability of at least one year at ambient temperature.

25. The method of claim 1 wherein the plant virus has been inactivated.

26. The kit according to claim 19, wherein the components are freeze-dried and contain a buffer for reconstitution.

27. The method of claim 1, wherein the plant virus has been inactivated by UV treatment.

28. An assay for the diagnosis of the presence or absence of a target nucleic acid molecule comprising the extraction control of claim 11.

29. A device for the extraction of RNA comprising an RNA extraction control.

30. The device according to claim 29, wherein said extraction control is a plant virus.

31. (canceled)

32. (canceled)

33. (canceled)

34. The composition of claim 15, wherein the plant virus has been inactivated.

35. The kit of claim 19, wherein the plant virus has been inactivated.

36. The composition of claim 34, wherein the plant virus has been inactivated by UV treatment.

37. The kit of claim 35, wherein the plant virus has been inactivated by UV treatment.

38. The method of claim 13 wherein the single-stranded positive RNA virus is selected from the genus tobamoviruses or topraviruses.

39. The method of claim 11 where the plant virus is tobacco mosaic virus or tobacco rattle virus.

40. The method according to claim 14 wherein the pathogen-derived RNA is of viral, bacterial, fungal, or parasitic origin.

41. The composition according to claim 17 wherein the positive single-stranded virus is selected from tobamoviruses or topraviruses.


SEQUENCE LISTING (3 sequences)

SEQ ID NO: 1 - DNA, 18 bp, Tobacco mosaic virus
gacgtgcgaattcctcag

SEQ ID NO: 2 - DNA, 21 bp, Tobacco mosaic virus
gttgatgttgcatactggttg

SEQ ID NO: 3 - DNA, 25 bp, Tobacco mosaic virus
aacctcttgaactgacagcgtgtgc